(19) United States
(12) Patent Application Publication
(10) Pub. No.: US 2020/0323938 A1
(43) Pub. Date: Oct. 15, 2020

(54) MANUFACTURE OF BOTANICALLY-DERIVED COMPOSITIONS

(71) Applicant: Evolve IP LLC, Spring, TX (US)

(72) Inventors: Ronald Eugene Theis, Spring, TX (US); Richard C. Knapp, III, Cypress, TX (US); Allen Fox, Robstown, TX (US); Michael John Richter, Robstown, TX (US)

(73) Assignee: Evolve IP LLC, Spring, TX (US)

(21) Appl. No.: 16/847,839

(22) Filed: Apr. 14, 2020

Related U.S. Application Data
(60) Provisional application No. 62/834,150, filed on Apr. 15, 2019.

(51) Int. Cl. A61K 36/185

(52) U.S. Cl. CPC A61K 36/185; A61K 2236/15; A61K 2236/17

(57) ABSTRACT

A method for manufacturing a botanically-derived composition includes mixing a cooling agent with a plant causing biomass particles to be removed from the plant. The method further includes creating a suspension comprising the biomass particles. The method further includes removing the biomass particles from the suspension. The method further includes drying the biomass particles to form the botanically-derived composition.

### CROSS-REFERENCE TO RELATED APPLICATIONS

This application claims the benefit of U.S. Provisional Application No. 62/834,150, filed Apr. 15, 2019 and titled “Manufacture of Botanically-Derived Compositions.”

### SUMMARY

A method for manufacturing a botanically-derived composition includes mixing a cooling agent with a plant causing biomass particles to be removed from the plant. The method further includes creating a suspension comprising the biomass particles. The method further includes removing the biomass particles from the suspension. The method further includes drying the biomass particles to form the botanically-derived composition.

A method for manufacturing a botanically-derived composition includes mixing a cooling agent with a plant causing most biomass particles to be removed from the plant. The method further includes infusing the plant with oil that captures remaining biomass particles. The method further includes pressing the plant to release the oil and form the botanically-derived composition.

A system of manufacturing a botanically-derived composition includes a suspender that mixes a cooling agent with a plant causing biomass particles to be removed from the plant, and that creates a suspension comprising the biomass particles. The system further includes a screening tower that removes the biomass particles from the suspension. The system further includes a dryer that dries the biomass particles to form the botanically-derived composition.

### BRIEF DESCRIPTION OF THE DRAWINGS

Systems and methods of manufacture of botanically-derived compositions are disclosed herein as well as the botanically-derived compositions themselves. In the drawings:

FIG. 1 is a system for manufacturing one or more botanically-derived composition in accordance with at least one illustrated embodiment;

FIG. 2 is a method for manufacturing a botanically-derived composition in accordance with at least one illustrated embodiment; and

FIG. 3 is a method for manufacturing a botanically-derived composition in accordance with at least one illustrated embodiment.

It should be understood, however, that the specific embodiments given in the drawings and detailed description thereto do not limit the disclosure. On the contrary, they provide the foundation for one of ordinary skill to discern the alternative forms, equivalents, and modifications that are encompassed together with one or more of the given embodiments in the scope of the appended claims.

### NOTATION AND NOMENCLATURE

Certain terms are used throughout the following description and claims to refer to particular system components and configurations. As one of ordinary skill will appreciate, companies may refer to a component by different names. This document does not intend to distinguish between components that differ in name but not function. In the following discussion and in the claims, the terms “including” and “comprising” are used in an open-ended fashion, and thus should be interpreted to mean “including, but not limited to . . . ”.

### DETAILED DESCRIPTION

The present disclosure relates to systems and methods for manufacturing botanically-derived compositions as well as the botanically-derived compositions themselves. The systems, methods, and compositions disclosed herein may use or be derived from many types of plants in various embodiments, and the resulting compositions may be used for various purposes. In at least one embodiment, the unprocessed plant from which the composition is derived is a cannabis plant including the biomass particles tetrahydrocannabinol (“THC”), cannabidiol (“CBD”), trichomes, and terpenes. In another embodiment, the unprocessed plant is a hemp plant with none of those biomass particles, but different biomass particles such as fibrous particles. In yet other embodiments, the unprocessed plants are known for their use as dyes or medicines, and the biomass particles that form the composition amplify such use. Finally, in other embodiments the biomass particles only include non-psychoactive cannabinoids. Here, THC refers to THCa or tetrahydrocannabinolic acid, which is non psycho-active because it has not been decarboxylated. For simplicity, the illustrated examples describe the systems, methods, and compositions using just one type of unprocessed plant, the cannabis plant, but such systems, methods, and compositions may be used for any type of unprocessed plant from which biomass particles may be removed.

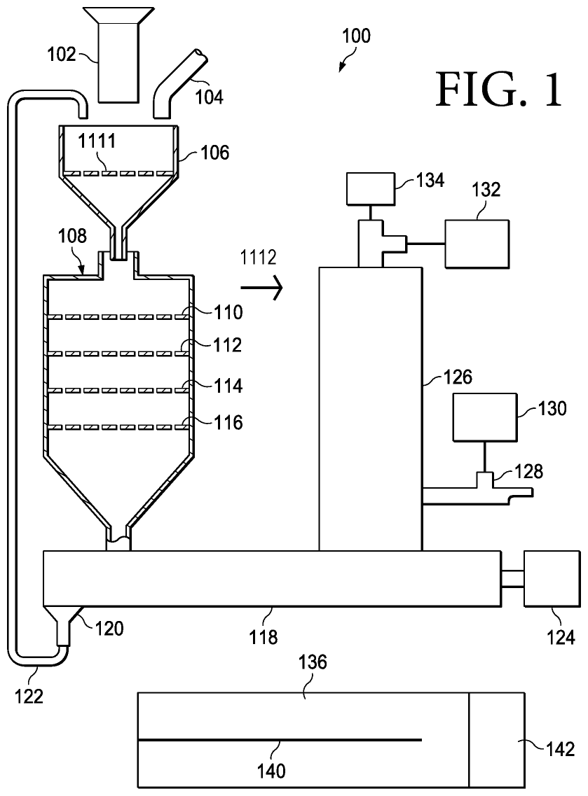

Turning to the figures, FIG. 1 illustrates a system 100 of manufacturing a botanically-derived composition in accordance with at least one embodiment. The system 100 includes a segmenter 102, a cooling conduit 104, a suspender 106 including a screen 1111 having holes of 190 micrometers or greater in an embodiment, a screening tower 108, a recirculation tank 118, a dryer 126, an infusion tank 136, a press 140, and a collection tank 142. The screening tower includes multiple stages of filtration such as screens 110, 112, 114, 116, and the recirculation tank 118 includes a chiller 124, a recirculating pump 120, and a recirculation pipe 122. The dryer 126 includes a nitrogen supply 132, an air blower 134, an air valve 128, and a vacuum generator 130.

The unprocessed plant is delivered to the segmenter 102, which segments the unprocessed plant. In an embodiment, the segmenter 102 includes multiple blades to cut the unprocessed plant into segments of roughly the same size and shape. For example, the segments are from 0.25 to 1 inches by from 0.25 to 1 inches. The segmenter 102 delivers the segments to the suspender 106 via gravity, pressure, or conveyer in various embodiments, and in a frozen, unfrozen, or flash frozen state in various embodiments.

The cooling conduit 104 delivers a cooling agent to the non-pressurized suspender 106. For example, the cooling conduit may be coupled to a cooling agent supply (not shown), and may deposit the cooling agent into the suspender 106 using gravity or pressure. In various embodiments, the cooling agent is water, ice, a slurry of water and ice, or the like. However, the cooling agent is not a solvent such as butane, other hydrocarbons, alcohol, oils, or carbon dioxide. The cooling agent cools or freezes biomass particles such as trichomes, cannabinoids (THC and CBD), and terpenes on or within the segments via direct or indirect contact. For example, the biomass particles may be cooled to 45° F. or below.

The suspender 106 creates a suspension including one or more types of biomass particles. In an embodiment, the suspender 106 separates biomass particles from the segments by mixing the cooling agent with the segments. The mixing may occur using a shaker drum or using physical agitators such as paddles in various embodiments. At a temperature of 45° F. or below, the biomass particles separate from the segments and become suspended in water. The suspension is delivered to the screening tower 108 using gravity or pressure. The screen 1111 may filter foreign particles and/or segments from the suspension. Once the suspension process is completed the segments are removed from the screen 1111 and delivered to the infusion tank 136. For example, the segments may be delivered by pipe, conveyer, by hand, or by gravity in various embodiments.

The screening tower 108 removes the biomass particles from the suspension. In an embodiment, the screening tower 108 includes several stages of removal, each stage removing a different type or size of biomass particle. For example, the screening tower 108 includes multiple configurable screens 110, 112, 114, 116 in an embodiment, each screen 110, 112, 114, 116 filtering various types or sizes of biomass particle from the suspension due to different sizes and/or shapes of holes in the screens. Specifically, different types of biomass particles may be different sizes and shapes, different strains of plants may have biomass particles of the same type but differing in size and shape, and even two or more of the same type of biomass particle on the same plant may be of different size and shape because each particle is in a different stage of growth. This wide variety of biomass particles may be accommodated efficiently using a rack system in which different screens may be added, removed, switched out, reordered, and the like. The first screen 110 may be used to remove unwanted foreign particles, rather than desired biomass particles, or may be used to capture the segments for later delivery to the infusion tank 136 if not performed at the suspender 106 stage.

The suspension passes through the screens 110, 112, 114, 116 using pressure, gravity, shaking, and the like alone or in various combinations in various embodiments. As can be appreciated, because the screens 110, 112, 114, 116 are reconfigurable and able to be removed, reordered, and swapped out with other screens within the screening tower 108 using the rack system, many configurations are possible. For simplicity, one example out of many possible combinations will be described. In an embodiment, the first screen 110 filters THC and CBD. This screen 110 may include holes from 160 to 190 micrometers (“microns”) in size. In an embodiment, the second screen 112 filters THC trichomes. This screen may include holes from 110 to 160 microns in size. In an embodiment, the third screen 114 filters trichomes, which are the primary components of botanically-derived compositions of the highest volume of THC. This screen may include holes from 45 to 110 microns in size. In an embodiment, the fourth screen 116 filters immature trichomes as well as terpenes, which is the primary component of a botanically-derived composition that over time, compiled, will have value. This screen may include holes 20 to 45 microns in size.

Such filtering by adhesion to the screens allow the different botanically-derived compositions to be separated and even further processed into different botanically-derived compositions. For example, the composition from screen 110 can be packaged for sale or combined with screen 112. Alternatively, the composition from screen 112 can be packaged for sale and/or combined with material in 110, 114 and/or 116, which can be used in the processing of edibles such as flours or sugars. Of course, the different (or differently sized) botanically-derived compositions filtered by the different screens may be processed and packaged separately as well in an embodiment.

The botanically-derived composition in screen 114 may be further processed into another botanically-derived composition named hashish using the system 100. For example, the contents of screen 114 are delivered to the dryer 126, which dries the THC material. Specifically, the screens 114 capturing THC material may be directly placed into the dryer 126, by hand or automated process such as a conveyor or articulated arm represented by arrow 1112 in FIG. 1, the dryer 126 accommodating the screens 114 using a similar rack system. If moved automatically, the dryer 126 may be situated much closer to the screening tower 108 than shown. For example they may be touching with a window between them to accommodate the moving screens. Alternatively, the THC material may be collected from the screens 114 and placed in the dryer 126 by hand or by machine. In an embodiment, the THC material is dried by removing greater than 50% of its water. For example, the dryer 126 may run dry air over the biomass particles for a specified amount based on the load to remove moisture by leaving valve 128 open. In an embodiment, the vacuum generator 130 regulates a vacuum pressure within the drying chamber of the dryer 126. Also, a low humidity air blower 134 and a nitrogen supply 132 supplies the drying chamber with gaseous nitrogen to enhance the drying process and keep the botanically-derived composition cool. The used nitrogen may be recirculated to the nitrogen supply 132. Once dry, the THC material is removed from the dryer, sealed, and packaged for sale or used for processing further into other items including, but not limited to edibles, flours, sugar, and the like.

After passing through the screening tower 108, the process fluid is delivered to a recirculation tank 118 using gravity, pressure, or the like. In an embodiment, the recirculation tank may hold a fluid capacity that is scalable to production capacity needed, which is chilled or kept cold using a chiller 124. The chiller may include a refrigeration unit that removes heat from the process fluid directly or indirectly, keeping the process fluid at or under 45° F. The recirculating pump 120 in or coupled to the recirculation tank 118 delivers the process fluid to the recirculation pipe 122, which routes the process fluid to the suspender 106.

The compositions remaining from screen 1111 and within 106 may be further processed into another botanically-derived composition, THC butter, using the system 100. The chamber of the infusion tank 136 may be heated and/or low-pressurized such that oil injected into the chamber turns into gas or a fine mist and infuses into the segments (delivered from the suspender 106 by hand or machine for example) to bond with or capture leftover biomass particles such as THC, CBD, trichomes, and terpenes. Next, a press 140 presses the segments to remove the infused oil. For example, the press 140 may include a screw press and plate to squeeze the infused oil out of the segments into a collection tank 142. The infused oil forms the botanically-derived composition and may be sealed and packaged for sale. Alternatively, the infused oil, may be used to form other botanically-derived compositions such as vape oils, shatters, flour, sugar, baking powder, butter, sugar, icing, and the like by combining the infused oil with other ingredients. This infusion and extraction process decarbs THCa to THC while still in the raw material plant mass.

Importantly, the described systems and methods do not use solvents such as butane, alcohol, or carbon dioxide, which cut the yield, concentration, effectiveness, and impact of the resultant compositions as well as introduce harmful chemicals and byproducts into the resultant compositions. Such solvents often change the state of the biomass particles into a liquid or gas, which further reduce yield, concentration, effectiveness, and impact. For example, a pressurized container, heat, and solvent process will begin the degradation process until the composition is degraded by as much as 50%. Also, such solvents also degrade THCa via decarboxylation. Additionally, by implementing the automation procedures described herein, the methods and systems scale to very large production volumes due to increases in efficiency and decreases in cost. These increases include manufacturing multiple different botanically-derived compositions from the same set of raw materials using the same system. Finally, the described systems and methods result in little to no waste products, can be produced at an extremely high volume, are not performed under high pressure, can be stored indefinitely, and take significantly less time by orders of magnitude compared to solvent-based methods that use butane, carbon dioxide, alcohol, and the like (for example, greater than 50% process time reduction in addition to significantly higher volumes).

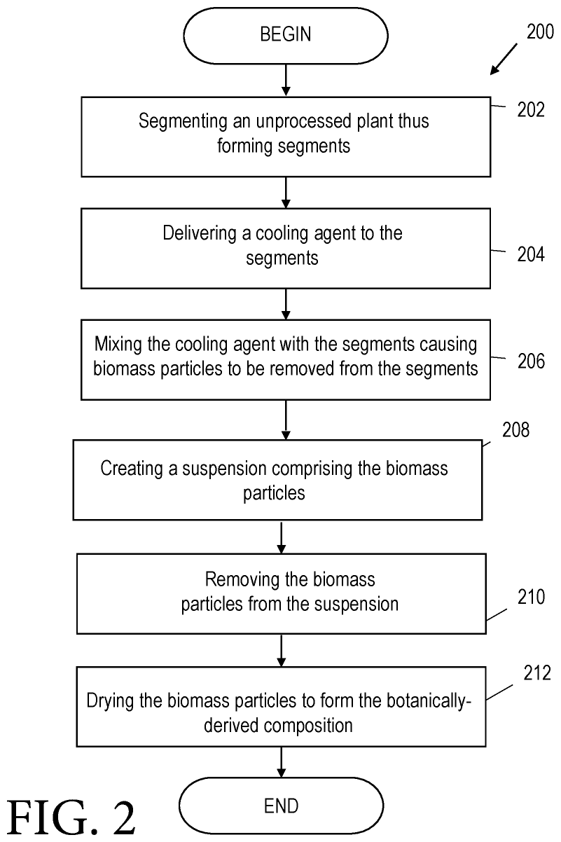

FIG. 2 illustrates a method 200 of manufacturing a botanically-derived composition in accordance with an embodiment. At 202, an unprocessed plant is segmented. For example, a segmenter as described above may be used. At 204, a cooling agent is delivered to the segments. For example, a cooling conduit as described above may be used. At 206, the cooling agent is mixed with the segments causing biomass particles to be removed from the segments. For example, a suspender as described above may be used. At 208, a suspension comprising the biomass particles is created. For example, a suspender as described above may be used. At 210, biomass particles are removed from the suspension. For example, a screening tower as described above may be used. At 212, the biomass particles are dried to form the botanically-derived composition. For example, a dryer as described above may be used.

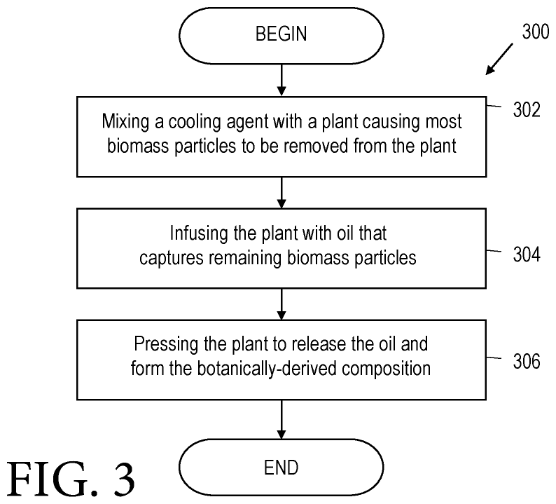

FIG. 3 illustrates a method 300 of manufacturing a botanically-derived composition in accordance with an embodiment. At 302, a cooling agent is mixed with a plant causing most of the biomass particles to be removed from the plant. For example, a suspender as described above may be used. At 304, the plant is infused with oil that captures the remaining biomass particles. For example, an infusion tank described above may be used. At 306, the plant is pressed to release the oil and form the botanically-derived composition. For example, a screw press and collection container as described above may be used.

In some aspects, apparatuses, systems, and methods for multiple games are provided according to one or more of the following examples:

### EXAMPLE 1

A method for manufacturing a botanically-derived composition includes mixing a cooling agent with a plant causing biomass particles to be removed from the plant. The method further includes creating a suspension comprising the biomass particles. The method further includes removing the biomass particles from the suspension. The method further includes drying the biomass particles to form the botanically-derived composition.

### EXAMPLE 2

A method for manufacturing a botanically-derived composition includes mixing a cooling agent with a plant causing most biomass particles to be removed from the plant. The method further includes infusing the plant with oil that captures remaining biomass particles. The method further includes pressing the plant to release the oil and form the botanically-derived composition.

### EXAMPLE 3

A system of manufacturing a botanically-derived composition includes a suspender that mixes a cooling agent with a plant causing biomass particles to be removed from the plant, and that creates a suspension comprising the biomass particles. The system further includes a screening tower that removes the biomass particles from the suspension. The system further includes a dryer that dries the biomass particles to form the botanically-derived composition.

The following features may be incorporated into the various embodiments described above, such features incorporated either individually in or conjunction with one or more of the other features: Drying the biomass particles may include drying the biomass particles using nitrogen and a vacuum generator. Removing the biomass particles may include removing different types of biomass particles from the suspension using a removable set of screens: a first screen that removes cannabinoids, a second screen that removes trichomes, and a third screen that removes terpenes. Creating the suspension and removing the biomass particles may be performed without solvents such as butane, alcohol, and carbon dioxide. Removing the biomass particles may include removing different types of biomass particles from the suspension in different stages. The different types of biomass particles may form different botanically-derived compositions. Removing the biomass particles may include removing different types of biomass particles from the suspension using a removable set of screens The screens may be automatically moved from a screening tower to a dryer. Infusing the plant may include directing the plant to an infusion tank, which may use low pressure and heats the air in the tank such that the oil is in a gaseous state. Infusing the plant may include circulating the plant within the tank. Pressing the plant may include heating the plant and pressing the plant using a screw press that empties into a collection container. The oil may be coconut oil, vegetable oil, or animal fat. The system may include a segmenter that segments an unprocessed plant and a cooling conduit that delivers the cooling agent to the segments for the mixing. The dryer may include a nitrogen supply, an air blower, and a vacuum generator. The screening tower may include a first rack for removable screens that remove the biomass particles, the dryer may include a second rack for the removable screens, and the system may include a conveyor to automatically move the screens from the first rack to the second rack. The screening tower may include removable screens that remove the biomass particles from the suspension: a first screen that removes cannabinoids, a second screen that removes trichomes, and a third screen that removes terpenes. The suspender and the screening tower may not use solvents such as butane, alcohol, or carbon dioxide. The system may include an infusion tank that receives the plant after the biomass particles are removed, infuses the plant with oil that captures remaining biomass particles, and presses the infused plant to release the oil and form another botanically-derived composition. The plant may be automatically delivered to the infusion tank after the biomass particles are removed.

Numerous other modifications, equivalents, and alternatives, will become apparent once the above disclosure is fully appreciated. For simplicity, the illustrated examples describe the systems, methods, and compositions using just one type of unprocessed plant, the cannabis plant, but such systems, methods, and compositions may be used for any type of unprocessed plant from which biomass particles may be removed. It is intended that the following claims be interpreted to embrace all such modifications, equivalents, and alternatives where applicable.

What is claimed is:

1. A method for manufacturing a botanically-derived composition comprising:
mixing a cooling agent with a plant causing biomass particles to be removed from the plant;
creating a suspension comprising the biomass particles;
removing the biomass particles from the suspension; and
drying the biomass particles to form the botanically-derived composition.

2. The method of claim 1, wherein drying the biomass particles comprises drying the biomass particles using nitrogen and a vacuum generator.

3. The method of claim 1, wherein removing the biomass particles comprises removing different types of biomass particles from the suspension using a removable set of screens, a first screen that removes cannabinoids, a second screen that removes trichomes, and a third screen that removes terpenes.

4. The method of claim 1, wherein creating the suspension and removing the biomass particles are performed without solvents such as butane, alcohol, and carbon dioxide.

5. The method of claim 1, wherein removing the biomass particles comprises removing different types of biomass particles from the suspension in different stages.

6. The method of claim 1, wherein the different types of biomass particles form different botanically-derived compositions.

7. The method of claim 1, wherein removing the biomass particles comprises removing different types of biomass particles from the suspension using a removable set of screens, the method further comprising automatically moving the screens from a screening tower to a dryer.

8. A method for manufacturing a botanically-derived composition comprising:
mixing a cooling agent with a plant causing most biomass particles to be removed from the plant;
infusing the plant with oil that captures remaining biomass particles; and
pressing the plant to release the oil and form the botanically-derived composition.

9. The method of claim 8, wherein infusing the plant comprises directing the plant to an infusion tank, which may use low pressure and heats the air in the tank such that the oil is in a gaseous state.

10. The method of claim 9, wherein infusing the plant comprises circulating the plant within the tank.

11. The method of claim 8, wherein pressing the plant comprises heating the plant and pressing the plant using a screw press that empties into a collection container.

12. The method of claim 8, wherein the oil is selected from the group consisting of: coconut oil, vegetable oil, and animal fat.

13. A system of manufacturing a botanically-derived composition comprising:
a suspender that mixes a cooling agent with a plant causing biomass particles to be removed from the plant, and that creates a suspension comprising the biomass particles;
a screening tower that removes the biomass particles from the suspension; and
a dryer that dries the biomass particles to form the botanically-derived composition.

14. The system of claim 13, further comprising:
a segmenter that segments an unprocessed plant; and
a cooling conduit that delivers the cooling agent to the segments for the mixing.

15. The system of claim 13, wherein the dryer comprises a nitrogen supply, an air blower, and a vacuum generator.

16. The system of claim 13, wherein the screening tower comprises a first rack for removable screens that remove the biomass particles, the dryer comprises a second rack for the removable screens, and the system further comprises a conveyor to automatically move the screens from the first rack to the second rack.

17. The system of claim 13 wherein the screening tower comprises removable screens that remove the biomass particles from the suspension, a first screen that removes cannabinoids, a second screen that removes trichomes, and a third screen that removes terpenes.

18. The system of claim 13, wherein neither the suspender nor the screening tower uses solvents such as butane, alcohol, or carbon dioxide.

19. The system of claim 13, further comprising an infusion tank that receives the plant after the biomass particles are removed, infuses the plant with oil that captures remaining biomass particles, and presses the infused plant to release the oil and form another botanically-derived composition.

20. The system of claim 19, wherein the plant is automatically delivered to the infusion tank after the biomass particles are removed.
